(19) United States
(12) Patent Application Publication
(10) Pub. No.: US 2003/0083211 A1
(43) Pub. Date: May 1, 2003

(54) Cosmetic bubble bath composition

(76) Inventors: Delphine Ribery, Levallois Perret (FR); Isabelle Penverne, Courbevoie (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/200,745

(22) Filed: Jul. 24, 2002

(30) Foreign Application Priority Data
Jul. 27, 2001 (FR) 01 10115

(51) Int. Cl. A61K 7/50; A61K 7/48

(52) U.S. Cl. 510/130; 510/135; 510/159; 510/424; 510/428

(57) ABSTRACT

A foaming cosmetic composition intended to be added to a bath, and comprising, in a cosmetically acceptable aqueous medium:

(a) an effective amount, such as 18% to 50%, by weight, relative to the total weight of the composition, of a lauryl ether sulfate of sodium, of potassium, of magnesium, of ammonium, of triethanolamine or of isopropanolamine or a mixture thereof;

(b) an effective amount, such as 0.01% to 2%, by weight, relative to the total weight of the composition, of a viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide, with the proviso that said composition is free of amphoteric or cationic surfactant.

The present invention relates, in an embodiment, to a cosmetic bubble bath composition comprising, in a cosmetically acceptable aqueous medium:

an effective amount of at least one lauryl ether sulfate chosen from lauryl ether sulfate of sodium, lauryl ether sulfate of potassium, lauryl ether sulfate of magnesium, lauryl ether sulfate of ammonium, lauryl ether sulfate of triethanolamine and lauryl ether sulfate of isopropanolamine, and

an effective amount of at least one viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide, with the proviso that said composition is free of amphoteric or cationic surfactant.

It is a known practice to add cosmetic products to bath water to form bubbles in the bath. Such products are generally known as “bubble baths”.

To satisfy consumers, bubble baths should produce a large volume of foam and the foam should be long-lasting, while collapsing as little as possible throughout the bathing time, i.e. usually 20 minutes.

However, most of the bubble baths known hitherto offer a satisfactory volume of foam at the beginning of the bath, but generally cannot maintain a satisfactory volume of foam after about 20 minutes bathing. The foam height in the bath can decrease very quickly, and the foam can finish by disappearing toward the end of the bath.

The bubble baths most commonly used hitherto comprise combinations of anionic surfactants, such as lauryl ether sulfate with amphoteric or cationic surfactants. This type of bubble bath cannot always provide an entirely satisfactory solution to the problem of the durability of the foam during bathing.

The aim of the present invention is to overcome the problem mentioned above.

The inventors have now discovered, surprisingly, novel bubble bath compositions that produce a large height of foam that can be sufficiently long-lasting throughout the bathing time. In addition, the foam produced by said composition can be at least one of soft, creamy, readily rinseable, nonsticky and well tolerated by the skin.

An embodiment of the present invention is thus a cosmetic bubble bath composition comprising, in a cosmetically acceptable aqueous medium:

(a) an effective amount of at least one lauryl ether sulfate chosen from lauryl ether sulfate of sodium, lauryl ether sulfate of potassium, lauryl ether sulfate of magnesium, lauryl ether sulfate of ammonium, lauryl ether sulfate of triethanolamine and lauryl ether sulfate of isopropanolamine; and

(b) an effective amount of at least one viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide,

with the proviso that said composition is free of amphoteric or cationic surfactant.

According to the present invention, the term “an effective amount” means an amount sufficient enough to compose a cosmetic bubble bath, which can produce a large height of foam that can be sufficiently long-lasting throughout the bathing time.

For example, an embodiment of the present invention is a cosmetic bubble bath composition comprising, in a cosmetically acceptable aqueous medium:

(a) from 18% to 50% by weight, relative to the total weight of the composition, of at least one lauryl ether sulfate chosen from lauryl ether sulfate of sodium, lauryl ether sulfate of potassium, lauryl ether sulfate of magnesium, lauryl ether sulfate of ammonium, lauryl ether sulfate of triethanolamine and lauryl ether sulfate of isopropanolamine, and

(b) from 0.01% to 2% by weight, relative to the total weight of the composition, of at least one viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide,

with the proviso that said composition is free of amphoteric or cationic surfactant.

Another embodiment of the present invention relates to a method of manufacturing a cosmetic bubble bath composition, including the composition according to one of the embodiments of the present invention.

In the composition according to one embodiment of the invention, the at least one lauryl ether sulfate is, for example, present in an amount ranging from 18% to 35% by weight relative to the total weight of the composition.

According to the present invention, for example, the at least one lauryl ether sulfate is sodium lauryl ether sulfate.

In the composition according to another embodiment of the invention, the at least one viscosifier is, for example, present in a concentration ranging from 0.1% to 1.5% by weight relative to the total weight of the composition.

Representative polyoxyethylenated fatty alcohols comprising from 30 to 200 mol of ethylene oxide, include: oxyethylenated (30 EO) cetyl alcohol, such as the product sold under the name NIKKOL BC-30TX® by the company Nikkol, oxyethylenated (50 EO) oleyl alcohol, such as the product sold under the name NIKKOL BO-50® by the company Nikkol, oxyethylenated (30 EO) behenyle alcohol, such as the product sold under the name NIKKOL BB-30® by the company Nikkol, and oxyethylenated (100 EO) stearyl alcohol, such as the product sold under the name BRIJ 700® by the company Uniquema.

Representative polyoxyethylenated fatty alkyl ethers comprising from 30 to 200 mol of ethylene oxide, include the compounds of general formula R—(OCH₂CH₂)ₙ—OR′, in which R denotes a C₁₀-C₃₀ alkyl radical, R′ denotes a C₁₀-C₃₀ alkyl radical substituted with a hydroxyl radical, and n is an integer ranging from 30 to 100, such as the product sold under the name ELFACOS GT 282 S® by the company Akzo; the compounds of general formula R—(OCH₂CH₂)ₙ—OR′ in which R denotes a C₁₆-C₁₈ alkyl radical, R′ denotes the alkyl radical C₁₄OH and n is equal to 60, commonly known under the CTFA Dictionary name of CETEARETH 60 MYRISTYL GLYCOL or HYDROGENATED TALLOWETH 60 MYRISTYL GLYCOL.

Representative polyoxyethylenated fatty esters comprising from 30 to 200 mol of ethylene oxide include PEG-150 dibehenate (CTFA name) such as the product sold under the name ETHOX PEG 6000 Dibehenate® by the company Ethox, polyethylene glycol palmitostearate (120 EO), such as the product sold under the name STEARATE 6000 WL 1644® by the company Gattefosse, and polyethylene glycol stearate (40 EO) such as the product sold under the name MYRJ 52® by the company Uniquema. Polyethylene glycol distearate 150 EO (CTFA name: PEG-150 distearate) can also be used, which is sold, for example, by the company Akzo under the name KESSCO PEG 6000 DS®, by the company Uniquema under the name ATLAS G-1821 V® or by the company CP Hall Company under the name EMULVIS®.

Representative alkanolamides include COCAMIDE MIPA (CTFA name), such as the product sold under the name EMPILAN CIS® by the company Albright & Wilson, or under the name REWOMID V3202® by the company Goldschmidt, the mixture of MYRISTAMIDE MEA and LAURAMIDE MEA (CTFA names), such as the product sold under the name NINOL LMP® by the company Stepan. Coconut fatty acid monoethanolamide (CTFA name: COCAMIDE MEA) can also be used, which is sold, for example, by the company Cognis under the name COMPERLAN 100®.

The cosmetically acceptable aqueous medium of the composition according to the invention generally comprises water or a mixture of water and at least one organic solvent. Organic solvents that may be used include polyols, for instance propylene glycol, glycerol, hexylene glycol, isoprene glycol, neopentyl glycol, polyethylene glycols or polyol ethers. A mixture of glycerol and hexylene glycol can also be used.

The at least one organic solvent may be present in proportions, for example, ranging from 0 to 20% by weight approximately relative to the total weight of the composition, and further, for example, ranging from 1% to 5% by weight approximately.

The composition according to the invention can further comprise at least one adjuvant chosen from opacifiers, thickeners, skin conditioners such as plant and mineral oils, waxes and ceramides, cationic and amphoteric polymers, silicones, wetting agents, such as sorbitol, preserving agents, pH agents, sequestering agents, colorants, fragrances, and fragrance peptizers.

The pH of the composition according to the invention is generally adjusted, for example, to about 4.5 to 7.5 by means of acidifying or basifying agents that are well known in cosmetics.

Among the basifying agents that may be mentioned, for example, are aqueous ammonia, alkali metal carbonates, alkanolamines such as monoethanolamine, diethanolamine, triethanolamine, and derivatives thereof, oxyethylenated and/or oxypropylenated hydroxyalkylamines and ethylenediamines, sodium hydroxide, potassium hydroxide and the compounds of the following formula:

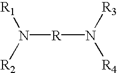

in which R is a propylene residue optionally substituted with a hydroxyl group or a C₁-C₄ alkyl radical; R₁, R₂, R₃ and R₄, which may be identical or different, are each chosen from a hydrogen atom, C₁-C₄ alkyl radicals and C₁-C₄ hydroxyalkyl radicals.

The acidifying agents are conventionally, for example, chosen from organic and mineral acids, and further such as citric acid, tartaric acid, and lactic acid.

Representative opacifiers that may be used in the composition according to the invention, include, for example, long-chain fatty alcohols, fatty esters of a polyol and fatty diethers, for example, distearyl ether, which are well known to those skilled in the art. An aqueous mixture of styrene-sodium acrylate copolymer, sodium lauryl sulfate, potassium sorbate, and polyethylene glycol (7 EO) tridecyl ether (CTFA name Trideceth-7), sold under the name OPACIFIER 680® by the company Rohm & Haas, can also be used.

Representative thickeners include nonionic cellulosic thickeners (such as hydroxyethylcellulose and hydroxypropylcellulose), the crosslinked polyacrylic acids sold under the name CARBOPOL®, and anionic, nonionic, cationic and amphoteric associative thickening polymers comprising at least one fatty chain; mention may also be made of sodium chloride, which can be, for example, used in a proportion of from 1% to 2% by weight relative to the total weight of the composition.

Needless to say, a person skilled in the art can take care to select at least one optional additional compound such that at least one of the properties intrinsically associated with the composition according to the invention are not, or are not substantially, adversely affected by the envisioned additions.

Another embodiment of the present invention relates to a cosmetic bubble bath composition comprising, in a cosmetically acceptable aqueous medium: an effective amount of a sodium lauryl ether sulfate, an effective amount of coconut fatty acid monoethanolamide, an effective amount of a mixture of glycerol and hexylene glycol, and an effective amount of sodium chloride, with the proviso that said composition is free of amphoteric or cationic surfactant.

For example, an embodiment of the present invention is a cosmetic bubble bath composition comprising, in a cosmetically acceptable aqueous medium: from 18% to 35% of a sodium lauryl ether sulfate, from 0.1% to 1.5% of coconut fatty acid monoethanolamide, from 1% to 5% of a mixture of glycerol and hexylene glycol, and from 1% to 2% of sodium chloride, the amounts being expressed by weight relative to the total weight of the composition, with the proviso that said composition is free of amphoteric or cationic surfactant.

The composition according to the invention can be, for example, in the form of a thickened liquid packaged in a bottle.

The example that follows illustrates the invention without being limiting in nature.

### EXAMPLE

The bubble bath composition below was prepared:

### Amounts Expressed as Grams of Active Material

| | |
|---|---|
| Sodium lauryl ether sulfate . . . | 21 |
| Cocamide MEA . . . | 1 |
| Glycerol . . . | 1 |
| Hexylene glycol . . . | 1 |
| Sodium chloride . . . | 1.8 |
| Opacifier (Opacifier 680 sold by | 0.7 |
| the company Rohm & Haas) | |
| Citric acid . . . | 0.7 |
| Fragrance, colorant, preserving agent . . . | q.s. |
| Demineralized water . . . q.s . . . | 100 |

20 grams of the composition as prepared were poured into a transparent bath, under the tap (38° C., constant flow, controlled hardness), and the bath was filled with about 100 liters of water.

With the bubble bath thus produced, a large foam height of about 18 cm at time zero was found, which lost only about 20% of its value after 20 minutes, whereas the bubble bath compositions of the prior art generally produce a foam which, after the same period, loses about 40% of its height.

What is claimed is:

1. A cosmetic bubble bath composition, comprising, in a cosmetically acceptable aqueous medium:
(a) 18% to 50% by weight, relative to the total weight of the composition, of at least one lauryl ether sulfate chosen from lauryl ether sulfate of sodium, lauryl ether sulfate of potassium, lauryl ether sulfate of magnesium, lauryl ether sulfate of ammonium, lauryl ether sulfate of triethanolamine and lauryl ether sulfate of isopropanolamine, and
(b) 0.01% to 2% by weight, relative to the total weight of the composition, of at least one viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide,
with the proviso that said cosmetic bubble bath composition is free of amphoteric or cationic surfactant.

2. A composition according to claim 1, wherein the at least one lauryl ether sulfate is present in a concentration ranging from 18% to 35% by weight relative to the total weight of the composition.

3. A composition according to claim 1, wherein the at least one viscosifier is present in a concentration ranging from 0.1% to 1.5% by weight relative to the total weight of the composition.

4. A composition according to claim 1, wherein the alkanolamides are chosen from coconut fatty acid monoethanolamides.

5. A composition according to claim 1, wherein the at least one lauryl ether sulfate is sodium lauryl ether sulfate.

6. A composition according to claim 1, wherein the aqueous medium comprises from 0 to 20% by weight of at least one organic solvent, relative to the total weight of the composition.

7. A composition according to claim 6, wherein the aqueous medium comprises from 1% to 5% by weight of at least one organic solvent, relative to the total weight of the composition.

8. A composition according to claim 6, wherein the at least one organic solvent is a mixture of glycerol and hexylene glycol.

9. A composition according to claim 1, further comprising at least one adjuvant chosen from opacifiers, thickeners, skin conditioners, wetting agents, preserving agents, pH agents, sequestering agents, colorants, fragrances and fragrance peptizers.

10. A composition according to claim 9, wherein the thickener is sodium chloride.

11. A composition according to claim 9, wherein the opacifiers are chosen from aqueous mixtures of styrene-sodium acrylate copolymer, sodium lauryl sulfate, potassium sorbate and polyethylene glycol (7 EO) tridecyl ether.

12. A composition according to claim 1, wherein the composition has a pH value ranging from 4.5 to 7.5.

13. A composition according to claim 1, wherein the composition is in the form of a thickened liquid packaged in a bottle.

14. A cosmetic bubble bath composition comprising, in a cosmetically acceptable aqueous medium: from 18% to 35% of a sodium lauryl ether sulfate, from 0.1% to 1.5% of coconut fatty acid monoethanolamide, from 1% to 5% of a mixture of glycerol and hexylene glycol, and from 1% to 2% of sodium chloride, the amounts being expressed by weight relative to the total weight of the composition, with the proviso that said cosmetic bubble bath composition is free of amphoteric or cationic surfactant.

15. A method of manufacturing a cosmetic bubble bath composition comprising including in the composition:
(a) 18% to 50% by weight, relative to the total weight of the composition, of at least one lauryl ether sulfate chosen from lauryl ether sulfate of sodium, lauryl ether sulfate of potassium, lauryl ether sulfate of magnesium, lauryl ether sulfate of ammonium, lauryl ether sulfate of triethanolamine and lauryl ether sulfate of isopropanolamine, and
(b) 0.01% to 2% by weight, relative to the total weight of the composition, of at least one viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide,
with the proviso that said composition is free of amphoteric or cationic surfactant.

16. A cosmetic bubble bath composition, comprising, in a cosmetically acceptable aqueous medium:
(a) an effective amount of at least one lauryl ether sulfate chosen from lauryl ether sulfate of sodium, lauryl ether sulfate of potassium, lauryl ether sulfate of magnesium, lauryl ether sulfate of ammonium, lauryl ether sulfate of triethanolamine and lauryl ether sulfate of isopropanolamine, and
(b) an effective amount of at least one viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide,
with the proviso that said cosmetic bubble bath composition is free of amphoteric or cationic surfactant.

17. A cosmetic bubble bath composition comprising, in a cosmetically acceptable aqueous medium: an effective amount of a sodium lauryl ether sulfate, an effective amount of coconut fatty acid monoethanolamide, an effective amount of a mixture of glycerol and hexylene glycol, and an effective amount of sodium chloride, with the proviso that said cosmetic bubble bath composition is free of amphoteric or cationic surfactant.

18. A method of manufacturing a cosmetic bubble bath composition comprising including in the composition:
(a) an effective amount of at least one lauryl ether sulfate chosen from lauryl ether sulfate of sodium, lauryl ether sulfate of potassium, lauryl ether sulfate of magnesium, lauryl ether sulfate of ammonium, lauryl ether sulfate of triethanolamine and lauryl ether sulfate of isopropanolamine, and
(b) an effective amount of at least one viscosifier chosen from (i) alkanolamides, (ii) polyoxyethylenated fatty alcohols comprising from 30 to 200 mol of ethylene oxide, polyoxyethylenated fatty alkyl ethers comprising from 30 to 200 mol of ethylene oxide, and polyoxyethylenated fatty esters comprising from 30 to 200 mol of ethylene oxide,
with the proviso that said composition is free of amphoteric or cationic surfactant.
